(19) United States
(12) Patent Application Publication
(10) Pub. No.: US 2025/0194671 A1
(43) Pub. Date: Jun. 19, 2025

(54) ULTRASONIC MIST INHALER

(71) Applicant: SHAHEEN INNOVATIONS HOLDING LIMITED, Abu Dhabi (AE)

(72) Inventors: Mohammed Alshaiba Saleh Ghannam Almazrouei, Abu Dhabi (AE); Imad Lahoud, Abu Dhabi (AE)

(21) Appl. No.: 19/071,651

(22) Filed: Mar. 5, 2025

Related U.S. Application Data
(63) Continuation of application No. 17/772,347, filed on Apr. 27, 2022, now Pat. No. 12,262,738.

(51) Int. Cl. A24F 40/05; A24B 15/167; A24F 40/10; A24F 40/42; A24F 40/485; A61M 11/00; B05B 17/00; B05B 17/06

(52) U.S. Cl. CPC A24F 40/05; A24B 15/167; A24F 40/10; A24F 40/42; A24F 40/485; B05B 17/0684; A61M 11/005; B05B 17/0646

(57) ABSTRACT

The invention relates to an ultrasonic mist inhaler, comprising a liquid reservoir structure comprising a liquid chamber adapted to receive liquid to be atomized, a sonication chamber in fluid communication with the liquid chamber; wherein the liquid reservoir structure comprises the sonication chamber as depicted in FIG. 3.

### CROSS-REFERENCE TO RELATED APPLICATIONS

This application is a continuation of application Ser. No. 17/772,347, filed Apr. 27, 2022; which is a National Phase Application under 35 U.S.C. § 371 of International Application No. PCT/IB2019/060806, filed Dec. 15, 2019, the entire contents of which are incorporated herein by reference.

### FIELD OF THE DISCLOSED TECHNOLOGY

The invention relates to an ultrasonic mist inhaler for atomizing a liquid by ultrasonic vibrations.

### BACKGROUND

Electronic vaporizing inhalers are becoming popular among smokers who also want to avoid the tar and other harsh chemicals associated with traditional cigarettes and who wish to satisfy the craving for nicotine. Electronic vaporizing inhalers may contain liquid nicotine, which is typically a mixture of nicotine oil, a solvent, water, and often flavoring. When the user draws, or inhales, on the electronic vaporizing inhaler, the liquid nicotine is drawn into a vaporizer where it is heated into a vapor. As the user draws on the electronic vaporizing inhaler, the vapor containing the nicotine is inhaled. Such electronic vaporizing inhalers may have medical purpose.

Electronic vaporizing inhalers and other vapor inhalers typically have similar designs. Most electronic vaporizing inhalers feature a liquid nicotine reservoir with an interior membrane, such as a capillary element, typically cotton, that holds the liquid nicotine so as to prevent leaking from the reservoir. Nevertheless, these cigarettes are still prone to leaking because there is no obstacle to prevent the liquid from flowing out of the membrane and into the mouthpiece. A leaking electronic vaporizing inhaler is problematic for several reasons. As a first disadvantage, the liquid can leak into the electronic components, which can cause serious damage to the device. As a second disadvantage, the liquid can leak into the electronic vaporizing inhaler mouthpiece, and the user may inhale the unvaporized liquid.

Electronic vaporizing inhalers are also known for providing inconsistent doses between draws. The aforementioned leaking is one cause of inconsistent doses because the membrane may be oversaturated or undersaturated near the vaporizer. If the membrane is oversaturated, then the user may experience a stronger than desired dose of vapor, and if the membrane is undersaturated, then the user may experience a weaker than desired dose of vapor. Additionally, small changes in the strength of the user's draw may provide stronger or weaker doses. Inconsistent dosing, along with leaking, can lead to faster consumption of the vaping liquid.

Additionally, conventional electronic vaporizing inhalers tend to rely on inducing high temperatures of a metal heating component configured to heat a liquid in the e-cigarette, thus vaporizing the liquid that can be breathed in. Problems with conventional electronic vaporizing inhalers may include the possibility of burning metal and subsequent breathing in of the metal along with the burnt liquid. In addition, some may not prefer the burnt smell caused by the heated liquid.

Electronic vaporizing inhalers are generally designed so that the liquid nicotine reservoir is arranged away from the metal heating component to prevent heating the unused liquid in the reservoir. This arrangement makes the inhaler device cumbersome and more complex to produce.

Thus, a need exists in the art for an electronic vaporizing inhaler that is better able to withstand these disadvantages.

### BRIEF SUMMARY

According to one aspect of the invention, an ultrasonic mist inhaler, comprises:
a liquid reservoir structure comprising a liquid chamber adapted to receive liquid to be atomized,
a sonication chamber in fluid communication with the liquid chamber,
wherein the liquid reservoir structure comprises the sonication chamber.

Using a sonication chamber for ultrasonic vibrations in an ultrasonic mist inhaler allows the combination of the liquid chamber and the sonication chamber into the liquid reservoir structure.

It is noted that the expression “mist” used in the invention means the liquid is not heated as usually in traditional inhalers known from the prior art. In fact, traditional inhalers use heating elements to heat the liquid above its boiling temperature to produce a vapor, which is different from a mist.

In fact, when sonicating liquids at high intensities, the sound waves that propagate into the liquid media result in alternating high-pressure (compression) and low-pressure (rarefaction) cycles, at different rates depending on the frequency. During the low-pressure cycle, high-intensity ultrasonic waves create small vacuum bubbles or voids in the liquid. When the bubbles attain a volume at which they can no longer absorb energy, they collapse violently during a high-pressure cycle. This phenomenon is termed cavitation. During the implosion very high pressures are reached locally. At cavitation, broken capillary waves are generated, and tiny droplets break the surface tension of the liquid and are quickly released into the air, taking mist form.

In the ultrasonic mist inhaler, the liquid reservoir structure may comprise an internal bore providing an air passage from the sonication chamber toward the surroundings.

In the ultrasonic mist inhaler, the liquid reservoir structure may comprise a peripheral inner chamber arranged between the liquid chamber and the internal bore, the peripheral inner chamber providing the fluid communication between the liquid chamber and the sonication chamber.

In the ultrasonic mist inhaler, the liquid reservoir structure may be made in one piece.

In the ultrasonic mist inhaler, the peripheral inner chamber may comprise an inner wall delimiting the peripheral inner chamber and the liquid chamber.

In the ultrasonic mist inhaler, the peripheral inner chamber may comprise an outer wall delimiting the peripheral inner chamber and the liquid chamber.

In the ultrasonic mist inhaler, the outer wall may comprise at least an aperture providing fluid communication between the liquid chamber and the peripheral inner chamber.

In the ultrasonic mist inhaler, the peripheral inner chamber may comprise a circular opening providing fluid communication between the peripheral inner chamber and the sonication chamber.

In the ultrasonic mist inhaler, the inner wall may extend in the sonication chamber.

In the ultrasonic mist inhaler, a capillary element may be arranged between the liquid chamber and the sonication chamber.

In the ultrasonic mist inhaler, the peripheral inner chamber may comprise the capillary element.

Such arrangement of the capillary element forms a wick and prevents uncontrolled leaking of liquid out of the liquid chamber.

Preferably, the capillary element is wrapped into the peripheral inner chamber around the internal bore.

The capillary element may be a gauze. The capillary element may be formed of bamboo fibers, preferably in 100% bamboo fibers, or cotton, silica, tobacco cotton, or any combination of such material.

In the ultrasonic mist inhaler, the capillary element may have a U-shape cross section.

In the ultrasonic mist inhaler, the capillary element may insert into the peripheral inner chamber from the circular opening.

In the ultrasonic mist inhaler, the sonication chamber comprises means of ultrasonic vibrations.

The expression “means of ultrasonic vibrations” is similar to the expression “ultrasonic oscillation component” used in the patent application PCT/IB2019/055192.

Preferably, the two lateral portion of the U-shape cross section are inserted into peripheral inner chamber and the central portion is in surface contact with the means of ultrasonic vibrations.

In the ultrasonic mist inhaler, the circular opening of the peripheral inner chamber may face the means of ultrasonic vibrations.

In the ultrasonic mist inhaler, the means of ultrasonic vibrations are supported by an elastic member.

The elastic member is formed from an annular plate-shaped rubber.

The elastic member has an inner hole wherein a groove is designed for maintaining the means of ultrasonic vibrations.

In this case, since the elastic member is in line contact with the means of ultrasonic vibrations, suppression of vibrations of the liquid reservoir structure can more effectively be prevented. Thus, fine particles of the liquid atomized by the atomizing member can be sprayed farther.

In the ultrasonic mist inhaler, a high-frequency voltage may be applied to the means of ultrasonic vibrations to ultrasonically vibrate the means of ultrasonic vibrations, whereby a liquid supplied to a portion of the means of ultrasonic vibrations can be atomized and sprayed.

According to the ultrasonic mist inhaler, fine particles of the liquid atomized by the means of ultrasonic vibrations having a relatively small size can be sprayed farther.

Such means of ultrasonic vibrations may be a transducer preferably designed in a circular plate-shape. The material of the piezoelectric transducer is preferably in ceramic.

In the ultrasonic mist inhaler, the internal bore may have a mechanical spring.

Preferably, the mechanical spring pushes the central portion of the capillary element onto the means of ultrasonic vibrations to ensure the surface contact.

In the ultrasonic mist inhaler, the sonication chamber may have a bottom plate closing the sonication chamber.

In the ultrasonic mist inhaler, the bottom plate may be configured to support the elastic member.

In the ultrasonic mist inhaler, the bottom plate may be arranged to receive connectors for powering the means of ultrasonic vibrations.

In the ultrasonic mist inhaler, a first connector and a second connector, the first connector is a spring-loaded contact probe and the second connector is a side pin crossing the elastic member. A spring-loaded contact probe provides a permanent contact with the means of ultrasonic vibrations.

Preferably, a metal plate ensures electrical contact between the second connector and the means of ultrasonic vibrations.

In the ultrasonic mist inhaler, the liquid reservoir structure may have a top end comprising an airway fluidically coupled to the internal bore and configured to allow liquid mist to flow out of the airway.

In the ultrasonic mist inhaler, the liquid reservoir structure may be arranged between a mouthpiece and a casing housing electrical components for powering and operating the inhaler.

The bottom plate is sealed, thus preventing leakage of liquid from the sonication chamber to the casing.

In the ultrasonic mist inhaler, the top end of the liquid reservoir structure may be coupled with the mouthpiece, the mouthpiece being configured to allow suction of liquid mist flowing out of the airway to the surroundings.

Advantageously, the liquid reservoir structure is detachable from the mouthpiece and the casing.

The liquid reservoir structure and the mouthpiece or the casing may include complimentary arrangements for engaging with one another; further such complimentary arrangements may include; a bayonet type arrangement; a threaded engaged type arrangement; a magnetic arrangement; and, a friction fit arrangement; wherein the liquid reservoir structure includes a portion of the arrangement and the mouthpiece or the casing includes the complimentary portion of the arrangement.

Such design permits the liquid reservoir structure to be disposable. The liquid reservoir structure may be removed and reinstalled when required; a feature that gives the user the freedom to interchange flavors without the limitation of either completely using or having to discard unused liquid.

Further, such design eliminates the risk of fatigue of the means of ultrasonic vibrations in the sonication chamber and has a disposable means of ultrasonic vibrations which prevents the risk of fatigue and a disposable capillary element which prevents the risk of mixing flavor.

In the ultrasonic mist inhaler, an integrated circuit may be coupled to the bottom end of the liquid reservoir structure and communicatively coupled to the means of ultrasonic vibrations, the integrated circuit configured to cause the means of ultrasonic vibrations to vibrate.

Preferably, the means of ultrasonic vibrations is in electrical communication with an integrated circuit, preferably via the connectors.

Advantageously, the integrated circuit is designed to convert a direct current into an alternate current at high frequency.

The means of ultrasonic vibrations may be powered from electrical components of the casing through the connectors.

Preferably, the casing is configured to contain an electrical storage device and may encase at least a portion of the integrated circuit.

The casing may have all features described in the patent application PCT/IB2019/055192.

The ultrasonic mist inhaler according to the invention, wherein said liquid to be received in the liquid chamber comprises 57-70% (w/w) vegetable glycerin and 30-43% (w/w) propylene glycol, said propylene glycol including nicotine and flavorings.

An ultrasonic mist inhaler or a personal ultrasonic atomizer device, comprising:
a liquid reservoir structure comprising a liquid chamber or cartridge adapted to receive liquid to be atomized,
a sonication chamber in fluid communication with the liquid chamber or cartridge,
wherein said liquid to be received in the liquid chamber comprises 57-70% (w/w) vegetable glycerin and 30-43% (w/w) propylene glycol, said propylene glycol including nicotine and flavorings.

### BRIEF DESCRIPTION OF THE DRAWINGS

Some embodiments are illustrated by way of example and not limitation in the figures of the accompanying drawings.

FIG. 1 is an exploded view of components of the ultrasonic mist inhaler according to an embodiment of the invention.

FIG. 2 is an exploded view of components of the inhaler liquid reservoir structure according to an embodiment of the invention.

FIG. 3 is a cross-section view of components of the inhaler liquid reservoir structure according to FIG. 1.

### DETAILED DESCRIPTION

The foregoing summary, as well as the following detailed description of certain embodiments of the present invention, will be better understood when read in conjunction with the appended drawings.

As used herein, an element recited in the singular and preceded with the word “a” or “an” should be understood as not excluding plural of said elements, unless such exclusion is explicitly stated. Furthermore, the references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

The present invention is directed to an ultrasonic mist inhaler. The description of the invention and accompanying figures will be directed to the electronic vaporizing inhaler embodiment; however, other embodiments are envisioned, such as an inhaler for hookah, flavored liquids, medicine, and herbal supplements. Additionally, the device can be packaged to look like an object other than a cigarette. For instance, the device could resemble another smoking instrument, such as a pipe, water pipe, or slide, or the device could resemble another non-smoking related object.

Ultrasonic mist inhalers are either disposable or reusable. The term “reusable” as used herein implies that the energy storage device is rechargeable or replaceable or that the liquid is able to be replenished either through refilling or through replacement of the liquid reservoir structure. Alternatively, in some embodiments reusable electronic device is both rechargeable and the liquid can be replenished. A disposable embodiment will be described first, followed by a description of a reusable embodiment.

Conventional electronic vaporizing inhaler tend to rely on inducing high temperatures of a metal component configured to heat a liquid in the inhaler, thus vaporizing the liquid that can be breathed in. The liquid typically contains nicotine and flavorings blended into a solution of propylene glycol (PG) and vegetable glycerin (VG), which is vaporized vi a heating component at high temperatures. Problems with conventional inhaler may include the possibility of burning metal and subsequent breathing in of the metal along with the burnt liquid. In addition, some may not prefer the burnt smell or taste caused by the heated liquid.

In contrast, aspects of the present disclosure include an ultrasonic mist inhaler that atomizes the liquid through ultrasonic vibrations, which produces micro water bubbles in the liquid. When the bubbles come into contact with ambient air molecules, water droplets of about 0.25 to 0.5 microns spray into the air, thereby generating micro-droplets that can be absorbed through breathing, similar to breathing in a mist.

No heating elements are involved, thereby leading to no burnt elements and reducing second-hand smoke effects.

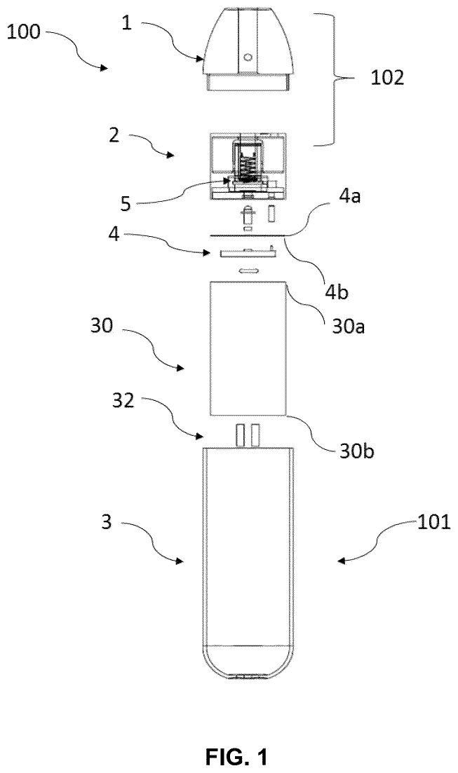

FIG. 1 depicts a disposable ultrasonic mist inhaler embodiment 100 of the invention. As can be seen in FIG. 1, the ultrasonic mist inhaler 100 has a cylindrical body with a relatively long length “L” as compared to the diameter “D.” In terms of shape and appearance, the ultrasonic mist inhaler 100 is designed to mimic the look of a typical cigarette. For instance, the inhaler can feature a first portion 101 that primarily simulates the tobacco rod portion of a cigarette and a second portion 102 that primarily simulates a filter. In the disposable embodiment of the invented device, the first portion and second portion are regions of a single, but-separable device. The designation of a first portion and a second portion is used to conveniently differentiate the components that are primarily contained in each portion.

As can be seen in FIG. 1, the ultrasonic mist inhaler comprises a mouthpiece 1, a liquid reservoir structure 2 and a casing 3. The first portion 101 comprises the casing 3 and the second portion 102 comprises the mouthpiece 1 and the reservoir structure 2.

The first portion 101 contains the power supply energy.

An electrical storage device 30 powers the ultrasonic mist inhaler 100. The electrical storage device 30 can be a battery, including but not limited to a lithium-ion, alkaline, zinc-carbon, nickel-metal hydride, or nickel-cadmium battery; a super capacitor; or a combination thereof. In the disposable embodiment, the electrical storage device 30 is not rechargeable, but, in the reusable embodiment, the electrical storage device 30 would be selected for its ability to recharge. In the disposable embodiment, the electrical storage device 30 is primarily selected to deliver a constant voltage over the life of the inhaler 100. Otherwise, the performance of the inhaler would degrade over time. Preferred electrical storage devices that are able to provide a consistent voltage output over the life of the device include lithium-ion and lithium polymer batteries.

The electrical storage device 30 has a first end 30a that generally corresponds to a positive terminal and a second end 30b that generally corresponds to a negative terminal. The negative terminal is extending to the first end 30a.

Because the electrical storage device 30 is located in the first portion 101 and the liquid reservoir structure 2 is located in the second portion 102, the joint needs to provide electrical communication between those components. In the present invention, electrical communication is established using at least an electrode or probe that is compressed together when the first portion 101 is tightened into the second portion 102.

In order for this embodiment to be reusable, the electrical storage device 30 is rechargeable. The casing 3 contains a charging port 32.

The integrated circuit 4 has a proximal end 4a and a distal end 4b. The positive terminal at the first end 30a of the electrical storage device 30 is in electrical communication with a positive lead of the flexible integrated circuit 4. The negative terminal at the second end 30b of the electrical storage device 30 is in electrical communication with a negative lead of the integrated circuit 4. The distal end 4b of the integrated circuit 4 comprise a microprocessor. The microprocessor is configured to process data from a sensor, to control a light, to direct current flow to means of ultrasonic vibrations 5 in the second portion 102, and to terminate current flow after a preprogrammed amount of time.

The sensor detects when the ultrasonic mist inhaler 100 is in use (when the user draws on the inhaler) and activates the microprocessor. The sensor can be selected to detect changes in pressure, air flow, or vibration. In a preferred embodiment, the sensor is a pressure sensor. In the digital embodiment, the sensor takes continuous readings which in turn requires the digital sensor to continuously draw current, but the amount is small and overall battery life would be negligibly affected.

Additionally, the integrated circuit 4 may comprise a H bridge, preferably formed by 4 MOSFETs to convert a direct current into an alternate current at high frequency.

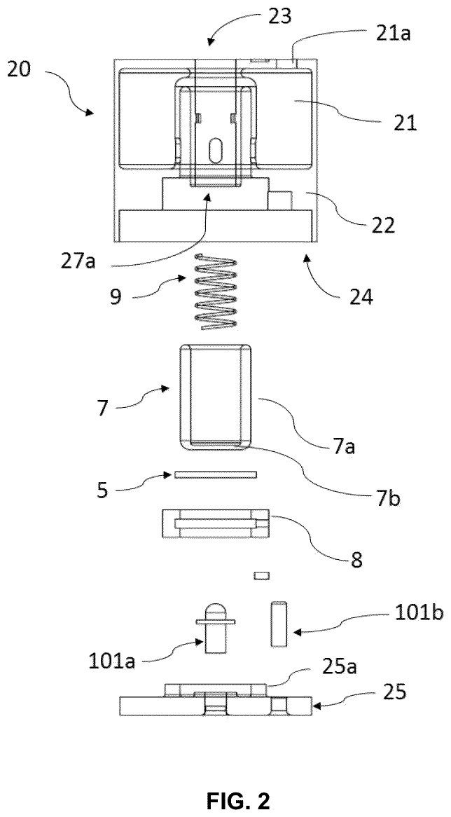

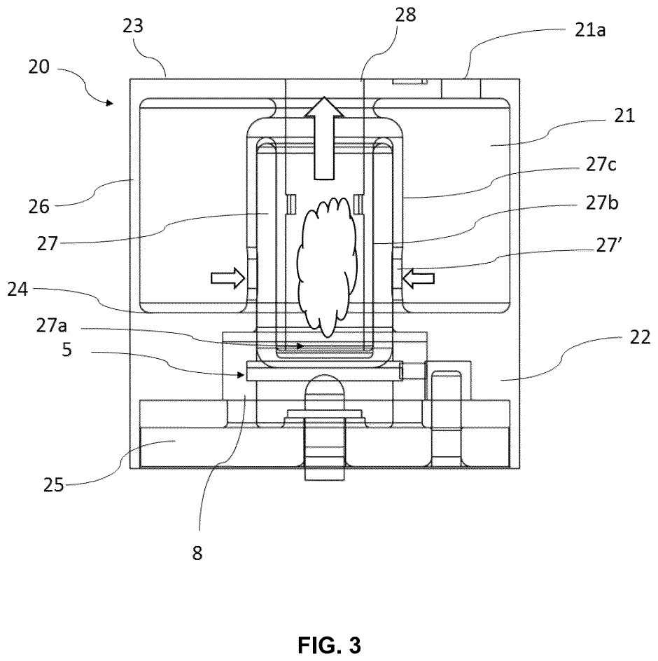

Referring to FIG. 2 and FIG. 3, illustrations of a liquid reservoir structure 2 according to an embodiment are shown. The liquid reservoir structure 2 comprises a liquid chamber 21 adapted to receive liquid to be atomized and a sonication chamber 22 in fluid communication with the liquid chamber 21.

In the embodiment shown, the liquid chamber 21 and sonication chamber 22 are part of a single assembly wherein a liquid is incorporated in the liquid chamber 21 from an inlet opening 21a.

As an example of sensor position, the sensor may be located in the sonication chamber.

The liquid reservoir structure 2 is arranged between the mouthpiece 1 and the casing 3 and is detachable from the mouthpiece 1 and the casing 3.

The liquid reservoir structure 2 and the mouthpiece 1 or the casing 3 may include complimentary arrangements for engaging with one another; further such complimentary arrangements may include one of the following: a bayonet type arrangement; a threaded engaged type arrangement; a magnetic arrangement; and, a friction fit arrangement; wherein the liquid reservoir structure 2 includes a portion of the arrangement and the mouthpiece 1 or the casing 3 includes the complimentary portion of the arrangement.

In the reusable embodiment, the components are substantially the same. The differences in the reusable embodiment vis-a-vis the disposable embodiment are the accommodations made to replace the liquid reservoir structure 2.

As shown in FIG. 3, the liquid reservoir structure 2 has a top wall 23, a middle wall 24 and a bottom wall 25 extending from a perimeter wall 26.

The top wall 23 and the middle wall 24 with the perimeter side 26 defines the outer of the liquid chamber 21. The middle wall 24 and the bottom wall 25 define with the perimeter wall 26 the outer of the sonication chamber 22. It means the middle wall 24 is a common wall with the liquid chamber 21 and the sonication chamber 22.

The liquid reservoir structure 2 has an internal bore 28 providing an air passage from the sonication chamber 21 toward the surroundings.

Further, the liquid reservoir structure 2 comprises of a peripheral inner chamber 27 arranged between the liquid chamber 21 and the internal bore 28. The peripheral inner chamber 27 provides the fluid communication between the liquid chamber 21 and the sonication chamber 22.

The peripheral inner chamber 27 has an outer wall 27c delimiting the peripheral inner chamber 27 and the liquid chamber 21. The peripheral inner chamber 27 further has an inner wall 27b delimiting the peripheral inner chamber 27 and the internal bore 28. More particularly, the internal bore 28 is formed by the inner wall 27b.

The outer wall 27c defines a plurality of transversely extending apertures 27′. The apertures 27′ create capillaries from the liquid chamber 21 through the thickness of the peripheral inner chamber 27 to the sonication chamber 22. The apertures 27′ can vary in diameter (the diameter of the capillary aperture will be dictated to a large extent by the viscosity of the fluid; a more viscous fluid can have a larger diameter capillary without leaking, while a less viscous fluid requires a smaller diameter capillary). Generally speaking, the diameters of the apertures 27′ are sufficient to facilitate passage of the not yet mist fluid. The apertures 27′ can be circular or any other of a variety of geometric shapes.

A circular opening 27a of the peripheral inner chamber 27 provides fluid communication between the peripheral inner chamber 27 and the sonication chamber 22.

The inner wall 27c of the peripheral inner chamber 27 extends beyond in the sonication chamber 22.

The top wall 23 of the liquid reservoir structure 2 comprises an airway fluidically coupled to the internal bore 28 and configured to allow liquid mist to flow out of the airway.

The top wall 23 of the liquid reservoir structure 2 may be coupled with the mouthpiece 1 and the mouthpiece 1 is configured to allow suction of mist liquid flowing out of the airway to the surroundings.

The bottom wall 25 of the liquid reservoir structure 2 is a bottom plate 25 closing the sonication chamber 22 so that the liquid reservoir structure 2 comprises the sonication chamber 22. The bottom plate 25 is sealed, thus preventing leakage of liquid from the sonication chamber 22 to the casing 3.

The peripheral inner chamber 27 defines a cavity. As depicted in FIG. 3, inserted into the cavity and in contact with the apertures 27′ is a capillary element 7. The capillary element 7 absorbs liquid from the liquid chamber 21 through the apertures 27′. The capillary element 7 is a wick. The capillary element 7 transports liquid to the sonication chamber 22 via capillary action. Preferably the capillary element 7 is made of bamboo fibers; however, cotton, paper, or other fiber strands could be used for a wick material.

In one embodiment of the ultrasonic mist inhaler 100, the capillary element 7 has a U-shape cross section with two lateral portion 7a inserted into peripheral inner chamber 27 and a central portion 7b in surface contact with the means of ultrasonic vibrations 5.

As can be seen in FIG. 3, the means of ultrasonic vibrations 5 are disposed directly below the capillary element 7.

The circular opening 27a of the peripheral inner chamber 27 faces the means of ultrasonic vibrations 5.

The means of ultrasonic vibrations 5 may be a transducer. For example, the means of ultrasonic vibrations 5 may be a piezoelectric transducer, preferably designed in a circular plate-shape. The material of the piezoelectric transducer is preferably in ceramic.

A variety of transducer materials can also be used for the means of ultrasonic vibrations 5.

The means of ultrasonic vibrations 5 are supported by an elastic member 8. The elastic member 8 is formed from an annular plate-shaped rubber having an inner hole 8′ wherein a groove is designed for maintaining the means of ultrasonic vibrations 5.

The bottom plate 25 has a protruding portion 25a on which the elastic member 8 is disposed so that the protruding 25a is inserted at least partially into the elastic member annular plate.

The bottom plate 25 is fixed to the liquid reservoir structure 2 by means of fixation such as screws, glue or friction.

As depicted in FIG. 3, the elastic member is in line contact with the means of ultrasonic vibrations 5 and prevents contact between the means of ultrasonic vibrations 5 and the inhaler walls, suppression of vibrations of the liquid reservoir structure are more effectively prevented. Thus, fine particles of the liquid atomized by the atomizing member can be sprayed farther.

The internal bore comprises a mechanical spring 9.

By pushing the capillary element 7b onto the means of ultrasonic vibrations 5, the mechanical spring 9 ensures a contact surface between them.

The means of ultrasonic vibrations 5 are in electrical communication with electrical contactors 101a, 101b. It is noted that, the distal end 4b of the integrated circuit 4 has an inner electrode and an outer electrode. The inner electrode contacts the first electrical contact 101a which is a spring contact probe, and the outer electrode contacts the second electrical contact 101b which is a side pin. Via the integrated circuit 4, the first electrical contact 101a is in electrical communication with the positive terminal of the electrical storage device 30 by way of the microprocessor, while the second electrical contact 101b is in electrical communication with the negative terminal of the electrical storage device 30.

The electrical contacts 101a, 101b crossed the bottom plate 25. The bottom plate 25 is designed to be received inside the perimeter wall 26 of the liquid reservoir structure 2. The bottom plate 25 rests on complementary ridges, thereby creating the sonication chamber 21.

The liquid reservoir structure 2 and the bottom plate 25 can be made using a variety of thermoplastic materials.

When the user draws on the ultrasonic mist inhaler 100, the liquid is drawn from the reservoir chamber 21 by capillarity, through the plurality of apertures 27′, and into the capillary element 7. The capillary element 7 brings the liquid into contact with the means of ultrasonic vibrations 5 of the inhaler 100. The user's draw also causes the pressure sensor to activate the integrated circuit 4, which directs current to the means of ultrasonic vibrations 5. Thus, when the user draws on the mouthpiece 1 of the inhaler 100, two actions happen at the same time. Firstly, the sensor activates the integrated circuit 4, which triggers the means of ultrasonic vibrations 5 to begin vibrating. Secondly, the draw reduces the pressure outside the reservoir chamber 21 such that flow of the liquid through the apertures 27′ begins, which saturates the capillary element 7. The capillary element 7 transports the liquid to the means of ultrasonic vibrations 5, which causes bubbles to form in a capillary channel by the means of ultrasonic vibrations 5 and mist the liquid. Then, the mist liquid is drawn by the user.

The ultrasonic mist inhaler 100 of the present disclosures is a more powerful version of current portable medical nebulizers, in the shape and size of current e-cigarettes and with a particular structure for effective vaporization. It is a healthier alternative to cigarettes and current e-cigarettes products.

The ultrasonic mist inhaler 100 of the present disclosures has particular applicability for those who use electronic inhalers as a means to quit smoking and reduce their nicotine dependency. The ultrasonic mist inhaler 100 provides a way to gradually taper the dose of nicotine.

Other embodiments of the invented ultrasonic mist inhaler 100 are easily envisioned, including medicinal delivery devices.

It is to be understood that the above description is intended to be illustrative, and not restrictive. For example, the above-described embodiments may be used in combination with each other. In addition, many modifications may be made to adapt a particular situation or material to the teachings of the invention without departing from its scope.

1. An ultrasonic mist inhaler, comprising:
a liquid reservoir structure comprising:
a top wall, a bottom wall, and a perimeter wall extending between the top wall and the bottom wall;
a liquid chamber defined by at least the top wall and the perimeter wall, the liquid chamber being adapted to receive liquid to be atomized;
a sonication chamber positioned adjacent to and in fluid communication with the liquid chamber;
an internal bore passing through the top wall and in communication with the sonication chamber, the internal bore providing an air passage from the sonication chamber toward the surroundings;
a peripheral inner chamber arranged between the liquid chamber and the internal bore, the peripheral inner chamber having an outer wall delimiting the peripheral inner chamber and the liquid chamber, and an inner wall delimiting the peripheral inner chamber and the internal bore, the internal bore being formed by the inner wall of the peripheral inner chamber; and
a capillary element positioned at least in part within the peripheral inner chamber, the capillary element configured to conduct liquid from the liquid chamber to the sonication chamber to provide the fluid communication between the liquid chamber and the sonication chamber.

2. The ultrasonic mist inhaler according to claim 1, wherein the liquid reservoir comprises a middle wall between the top wall and the bottom wall, wherein the top wall of the liquid reservoir and the middle wall with the perimeter side defines the outer wall of the liquid chamber.

3. The ultrasonic mist inhaler according to claim 2, wherein the middle wall and the bottom wall define with the perimeter wall the outer of the sonication chamber.

4. The ultrasonic mist inhaler according to claim 2, wherein the middle wall is a common wall with the liquid chamber and the sonication chamber.

5. The ultrasonic mist inhaler according to claim 4, wherein the outer wall comprises at least an aperture providing fluid communication between the liquid chamber and the peripheral inner chamber.

6. The ultrasonic mist inhaler according to claim 1, wherein the peripheral inner chamber comprises a circular opening providing fluid communication between the peripheral inner chamber and the sonication chamber.

7. The ultrasonic mist inhaler according to claim 6, wherein the inner wall extends in the sonication chamber.

8. The ultrasonic mist inhaler according to claim 1, wherein the liquid reservoir structure is made in one piece.

9. The ultrasonic mist inhaler according to claim 1, wherein the sonication chamber comprises an ultrasonic vibration device.

10. The ultrasonic mist inhaler according to claim 9, wherein the ultrasonic vibration device is supported by an elastic member.

11. The ultrasonic mist inhaler according to claim 1, wherein the internal bore has a mechanical spring.

12. The ultrasonic mist inhaler according to claim 1, wherein the liquid reservoir structure comprises a top end comprising an airway fluidically coupled to the internal bore and configured to allow liquid mist to flow out of the airway.

13. The ultrasonic mist inhaler according to claim 1, the liquid reservoir structure is arranged between a mouthpiece and a casing housing electrical components for powering and operating the inhaler.

14. The ultrasonic mist inhaler according to claim 1, wherein the liquid chamber contains a liquid comprising 57-70% (w/w) vegetable glycerin and 30-43% (w/w) propylene glycol, the propylene glycol including nicotine and flavorings.
